(19) United States
(12) Patent Application Publication
(10) Pub. No.: US 2023/0346995 A1
(43) Pub. Date: Nov. 2, 2023

(54) USE OF ELECTRIC CURRENT OR FIELD TO MANAGE RISK OF INFECTION BY ANTIMICROBIAL-RESISTANT MICROORGANISMS

(71) Applicant: THE TRUSTEES OF INDIANA UNIVERSITY, Bloomington, IN (US)

(72) Inventors: Chandan K. SEN, Indianapolis, IN (US); Sashwati ROY, Indianapolis, IN (US)

(21) Appl. No.: 17/923,056

(22) PCT Filed: May 11, 2021
(86) PCT No.: PCT/US2021/031681

§ 371 Date: Nov. 3, 2022

Related U.S. Application Data
(60) Provisional application No. 63/024,198, filed on May 13, 2020.

(51) Int. Cl. A61L 2/03; A61K 33/38; A61K 33/28; A61K 31/496; A61P 31/10; A61P 31/04; A61N 1/40

(52) U.S. Cl. CPC A61L 2/03; A61K 33/38; A61K 33/28; A61K 31/496; A61P 31/10; A61P 31/04; A61N 1/40; A61L 2202/21

(57) ABSTRACT

Compositions and methods are provided for treating an article that is susceptible to contamination by an antimicrobial-resistant microorganism, wherein the susceptible article is exposed to an optimized electric current or field. Methods to treat an individual in need of therapy are also provided, including for example, individuals having a bacterial, viral, fungal, or parasitic infection, or individuals having an antimicrobial-resistant microbial infection, e.g., in a wound, by applying a predetermined electric current to the individual. The methods disclosed herein can be used in conjunction with a therapeutic pharmaceutical composition comprising an antimicrobial agent such as an antifungal or antibiotic pharmaceutical.

### CROSS REFERENCE TO RELATED APPLICATIONS

This application claims priority to the following: U.S. Provisional Patent Application No. 63/024,198 filed on May 13, 2020, the disclosure of which is expressly incorporated herein.

### BACKGROUND OF THE DISCLOSURE

New strains and species of antimicrobial-resistant microorganisms or microbes (AMR) are increasingly prevalent. The increased emergence of AMR is a growing public health crisis; they cause a staggering number of infections and deaths, impair the success of surgical procedures including orthopedic prosthetic surgeries. The onset of antibiotic resistance is becoming widespread among numerous microbes that infect humans.

Microorganisms are continuously gaining mechanisms of antimicrobial resistance despite new generations of antimicrobials. With this comes higher health care costs, poorer outcomes, and a higher risk of mortality among those infected with resistant strains. The pressure to use antimicrobials, and the fact that their resistance-producing effects cannot be reversed or undone, will ensure that the multidrug-resistant microbes (MDRM) increase in number, be persistent, and evolve more rapidly than new generations of antimicrobials can be discovered. In the United States there are an estimated 35,000 deaths and more than 2.8 million illnesses resulting from resistant organisms. Direct costs exceed $20 billion annually, and indirect costs exceed $35 billion.

Accordingly there is a need for additional therapeutic tools for use in combination with existing treatment strategies for combating infections caused by antibiotic resistant microorganisms.

### SUMMARY

The present disclosure is directed to composition and methods of providing a novel application of chemoelectrical intervention (CEI) to treat antimicrobial resistant (AMR). microbes In one embodiment, energy, in the form of electric field or current, is provided therapeutically or prophylactically to a patient or a medical device, implant or instrument to render AMR microbes less pathogenic. For example, the AMR microbe may become more susceptible to antimicrobial drugs to which they may be otherwise resistant and/or may become less pathogenic. This principle applies across multiple pathogenic microbes ranging from bacteria to yeast and viruses that are of relevance to human disease.

The invention employs a novel biophysical approach that may be effective on its own or may complement the conventional biopharmaceutical approaches to treat and manage infections. The invention defines a new paradigm for management of antimicrobial resistance (AMR). The inventive method exerts a relatively weak electric field or current of specific strength, ranging from 10 V/cm to 100 V/cm, sufficient to manage risk of infection by providing at least microbial static effects, i.e., blocking their multiplication, and in some cases providing microbial cidal effects, i.e., killing them. In one non-limiting embodiment, the method applies an electric field or current to which the microbes are sensitive using a wireless electroceutical device or dressing (WED).

In one embodiment the electroceutical device comprises a fabric of synthetic fibers that comprises a pattern of alternating metals that form an appropriate oxidation-reduction (redox) couple for generating an electric field or current due to the transfer of an electron from one metal to the other when contacted with an aqueous solution, and in the absence of a power source. In one embodiment the electroceutical device comprises alternating dots of silver and zinc as the redox couple. In one embodiment the electroceutical device comprises a fabric of synthetic fibers having Ag dots (1-2 mm) and Zn dots (1-2 mm) printed on the fabric in proximity of about 0.5, 1, 1.5 or 2 mm to each other. In one embodiment the electroceutical device comprises a fabric of synthetic fibers having Ag dots (of about 2 mm) and Zn dots (of about 1 mm) printed on the fabric in proximity of about 0.5, 1, or 1.5 to each other. In one embodiment the electroceutical device comprises an FDA approved silver-zinc coupled bioelectric dressing (BED) which is currently being used in clinical wound care. Such a device is commercially available from Vomaris Innovations, Inc. (Tempe, AZ). The advantage of this device is that it is wireless and has no need for an external power source, can be cut to any desired shape and size, conforms to irregular surfaces, and provides an electrical field in the range of the physiologic fields (Banerjee et al, PLoS One 9(3); 2014). FDA approved silver-zinc coupled bioelectric dressing (BED) which is currently being used in clinical wound care. In one embodiment, the method is used in combination with conventional biopharmaceutical approaches.

In one embodiment, the method is used independently of biopharmaceutical approaches, either in whole or in part.

The invention has utility to treat wound infections as well as to manage the risk of infected hardware (catheters, implants, surgical tools, etc.) without adversely affecting living human cells. The invention has utility to cleanse hospital facilities and fabrics of well-known hospital-AMR pathogens. Other public facilities can be cleansed using appropriate devices dispensing weak electric fields or current for short periods of time. The method is not limited to use with bacteria and may be used with other microbes. In one embodiment the method is used with viruses. Viruses are often charged and utilize electrostatic forces to attach to surfaces and multiply. Disruption of these biophysical properties limits viral infectivity. In one embodiment the method is used with fungi such as yeast. Fungi contain three cell wall components, two of which are essential for pathogenesis while the third is required for protection. In one embodiment, the application of the relatively weak electric field or current, while desirably providing a static effect to microbes, enhances the immune system thereby producing a synergistic therapeutic effect.

In accordance with one embodiment a method of rendering AMR microbes less pathogenic and/or enhancing the efficacy of antimicrobial agents against an antimicrobial resistant strain is provided. In one embodiment the method comprises applying an electric current or field to the antimicrobial resistant strain. In one embodiment the applied electric field is in the range of about 10 V/cm to about 100 V/cm, 1 V/cm to about 50 V/cm, or 25 V/cm to about 50 V/cm. The electrical field can be generated using any technique known to the skilled practitioner including using a wireless electroceutical device (WED) or the use of a large electromagnetic coil. In one embodiment the electric current or field is applied as a series of pulses. In one embodiment the AMR strain is contacted with an antimicrobial agent during an application of, or between a first and second administered dose of, the electric current or field. In one embodiment the AMR strain is contacted with said antimicrobial agent after application of the electric current or field has been completed. In one embodiment the AMR strain is a fungus and the antimicrobial agent is a fungicide. In one embodiment the AMR strain is an antibiotic resistant bacteria and the antimicrobial agent is an antibiotic. In one embodiment the AMR strain is present on a surface of an inanimate object and in another embodiment the AMR strain is present in an infected tissue of a patient, and said method comprises applying the electric current or field to said infected tissue.

In accordance with one embodiment a method of reducing the risk of contamination by a pathogenic microorganism on an article is provided wherein the method comprises applying an electric current or field to the article, optionally in conjunction with application of an antimicrobial agent.

In accordance with one embodiment a method of treating a subject having an antimicrobial-resistant microbial infection is provided wherein the method comprises applying an electric current or field to the infected tissues to facilitate recovery from said infection in the subject, optionally in conjunction with application of an antimicrobial agent.

Table I provides a list of AMR bacteria, their associated pathology, and sensitivity to an electric field (presented by a wireless electroceutical device, WED).

TABLE 1

| | | WED |
|---|---|---|
| Organism | Pathological Manifestation | Sensitivity |
| Imipenem resistant Acinetobacter | Health care associated infections, wound | High |
| baumannii (BAA-1605) | infections, bacteriamia | |
| Imipenem resistant Pseudomonas | Soft tissue infections, systemic infection | High |
| aeruginosa (BAA-2108) | in burn patients. | |
| Ertipenem resistant Klebsiella | Pneumonia, urinary tract infections, | High |
| pneumoniae (BAA-2342) | intro-abdominal infections | |
| Ampicillin resistant Salmonella | Typhoid fever, food poisoning, | High |
| enterica (ATCC-19214) | gastroenteritis | |
| Methicillin resistant Staphylococcus | Skin infections, osteomyelitis, toxic | High |
| aureus (BAA 1695) | shock syndrome | |

### BRIEF DESCRIPTION OF THE DRAWINGS

FIG. 1 is a bar graph providing data from a viability assessment (optical density) by standard growth curve using a planktonic model for the imipenem-resistant Acinetobacter baumannii (A. baumannii) (BAA-1605).

FIG. 2 is a bar graph providing data from a viability assessment (concentration) by standard growth curve using a planktonic model for the imipenem-resistant A. baumannii (BAA-1605). Lane 1: no treatment; Lane 2: imipenem; Lane 3: Ag dots; Lane 4: WED; Lane 5: imipenem+Ag dots; Lane 6: WED+imipenem.

FIG. 3 is a bar graph providing data from a viability assessment (optical density) by standard growth curve using a planktonic model for the imipenem-resistant Pseudomonas aeruginosa (P. aeruginosa) (BAA-2108). Lane 1: no treatment; Lane 2: imipenem; Lane 3: Ag dots; Lane 4: WED; Lane 5: imipenem+Ag dots; Lane 6: WED+imipenem.

FIG. 4 is a bar graph providing data from a viability assessment (concentration) by standard growth curve using a planktonic model for the imipenem-resistant P. aeruginosa (BAA-2108).

FIG. 5 is a bar graph providing data from a viability assessment (optical density) by standard growth curve using a planktonic model for ertapenem-resistant Klebsiella pneumoniae (K. pneumonia) (BAA-2342). Lane 1: no treatment; Lane 2: ertapenem; Lane 3: Ag dots; Lane 4: WED; Lane 5: ertapenem+Ag dots; Lane 6: WED+ertapenem.

FIG. 6 is a bar graph providing data from a viability assessment (concentration) by standard growth curve using a planktonic model for the ertapenem-resistant K. pneumonia (BAA-2342). NG=no growth.

FIG. 7 is a bar graph providing data from a viability assessment (optical density) by standard growth curve using a planktonic model for the ampicillin-resistant Salmonella enterica (S. enterica) (ATCC-19214). Lane 1: no treatment; Lane 2: ampicillin; Lane 3: Ag dots; Lane 4: WED; Lane 5: ampicillin+Ag dots; Lane 6: WED+ampicillin.

FIG. 8 is a bar graph providing data from a viability assessment (optical density) by standard growth curve using a planktonic model for the methicillin-resistant Staphylococcus aureus (S. aureus) (BAA-1695). Lane 1: no treatment; Lane 2: methicillin; Lane 3: Ag dots; Lane 4: WED; Lane 5: methicillin+Ag dots; Lane 6: WED+methicillin.

FIG. 9 is a bar graph providing data from a viability assessment (concentration) by standard growth curve using a planktonic model for the methicillin-resistant S. aureus (BAA-1695).

FIG. 10 is a bar graph providing data from the results of real-time PCR analysis of bacterial biofilm and virulence-related genes: qPCR analysis for the expression of A. baumanii bfm-s and bap in response to silver dots and WED. Lane 1: no treatment; Lane 2: Ag dots; Lane 3: WED.

FIG. 11 is a bar graph providing data from the results of real-time PCR analysis of bacterial biofilm and virulence-related genes: qPCR analysis for the expression of P. aeruginosa lasA, mexA and toxA in response to silver dots and WED. Lane 1: no treatment; Lane 2: Ag dots; Lane 3: WED.

FIG. 12 is a bar graph providing data from the results of real-time PCR analysis of bacterial biofilm and virulence-related genes: qPCR analysis for the expression of S. aureus (MRSA) agrA and RNAIII in response to silver dots and WED. Lane 1: no treatment; Lane 2: Ag dots; Lane 3: WED.

FIG. 13 is a bar graph providing data from the results of real-time PCR analysis of bacterial biofilm and virulence-related genes: qPCR analysis for the expression of K. pneumoniae blaKPC and fimH in response to silver dots and WED. N=4, Mean±SEM, * represents p value<0.05. Lane 1: no treatment; Lane 2: Ag dots; Lane 3: WED.

FIG. 14 is a bar graph providing data from the results of real-time PCR analysis of bacterial biofilm and virulence-related genes: qPCR analysis for the expression of S. enterica csgA and csgD in response to silver dots and WED. Lane 1: no treatment; Lane 2: Ag dots; Lane 3: WED.

FIG. 15 is a bar graph providing data from the viability assessment by the standard growth curve of Candida albicans (C. albians) (planktonic mode). Lane 1: Control; Lane 2: unprinted dressing; Lane 3: Ag dressing; Lane 4: WED; Lane 5: ketoconazole; Lane 6: WED+ketoconazole.

FIG. 16 Candida albicans cells were treated with Ag—Zn dressing or WED, alone or in combination with ketoconazole and stained with two fluorescent dyes, DiBAC4(3) and PI. FIG. 16 is a bar graph providing data from flow cytometry analysis indicating the percentage of dual stained populations resulting from the indicated treatments. The data indicates that that WED, alone or in combination with ketoconazole, caused cell membrane damage. Lane 1: no treatment; Lane 2: unprinted dressing; Lane 3: Ag dressing; Lane 4: WED; Lane 5: ketoconazole; Lane 6: WED+ketoconazole.

FIG. 17 is a bar graph providing data from an ultrastructure analysis for cell wall thickness (C. albicans). C. albicans cells treated with WED in combination with ketoconazole showed an increase in cell wall thickness by two-fold (˜300 nm) compared to untreated cells (˜150 nm), and the outer cell wall consisting of mannan residues was absent in WED+ketoconazole treated cells.

FIG. 18A-18D shows the effect of wound dressings on planktonic growth of C. auris isolates 0381 (FIG. 18A), 0382 (FIG. 18B), 0383, (FIG. 18C) and 0384 (FIG. 18D) found to be highly susceptible to treatment with WED alone or in combination with amphotericin B when compared to planktonic growth of cells treated only with amphotericin B.

FIGS. 19A-19D shows the effect of wound dressings on the planktonic growth of C. auris isolates 0385 (FIG. 19A), 0386 (FIG. 19B), 0387 (FIG. 19C), and 0388 (FIG. 19D), found to be highly susceptible to treatment with WED alone or in combination with amphotericin B compared to planktonic growth of cells treated only with 1amphotericin B.

FIG. 20A & 20B shows the effect of wound dressings on the planktonic growth of C. auris isolates 0389 (FIG. 20A) and 0390 (FIG. 20B). C. auris 0389 was resistant to silver or zinc only dressing while it was susceptible when treated with WED alone or in combination with amphotericin B. C. auris 0390 was susceptible when treated with silver or zinc only dressing as well as WED alone or in combination with amphotericin B.

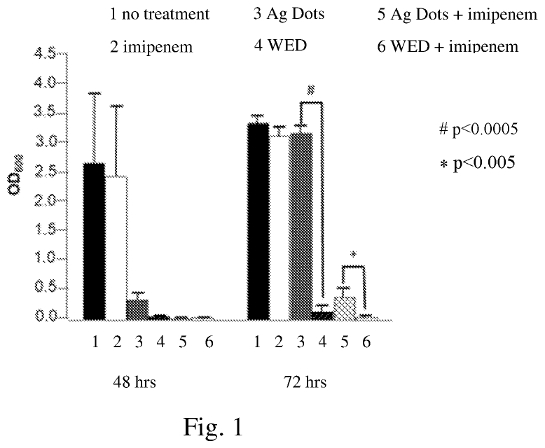

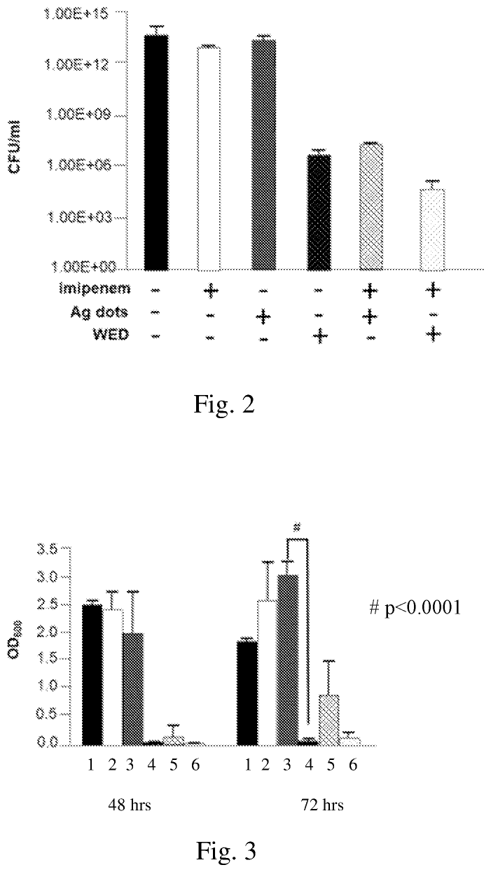

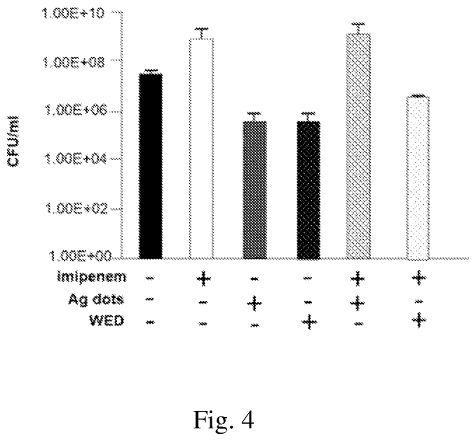

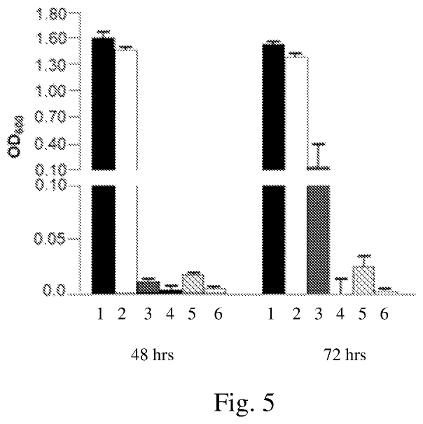

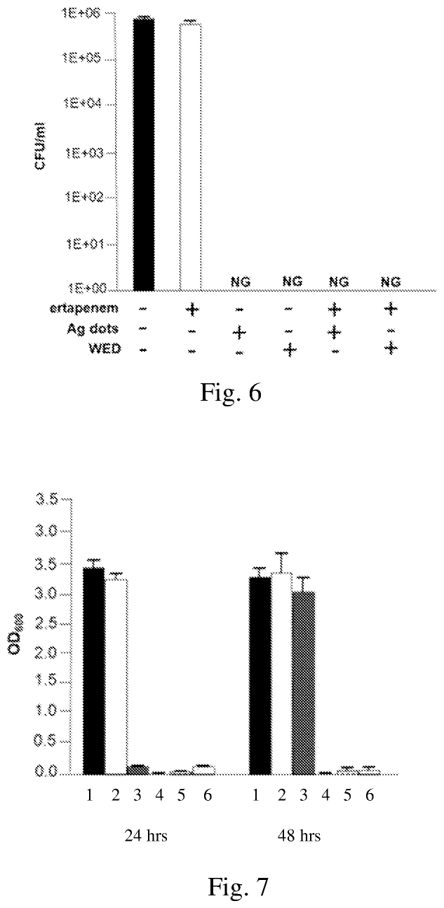

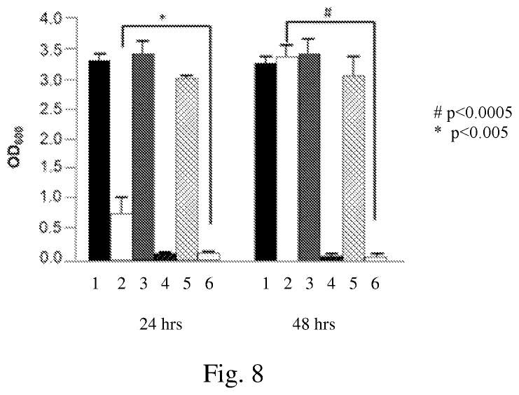

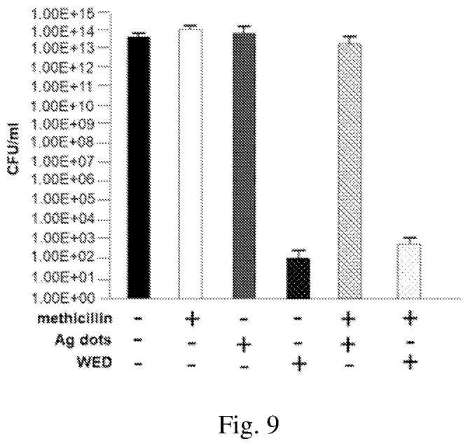

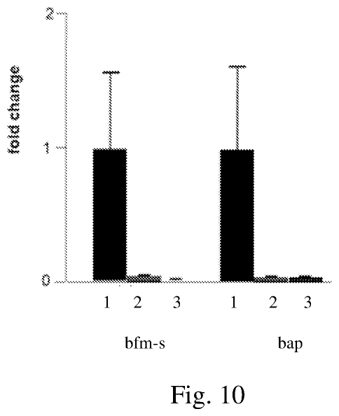

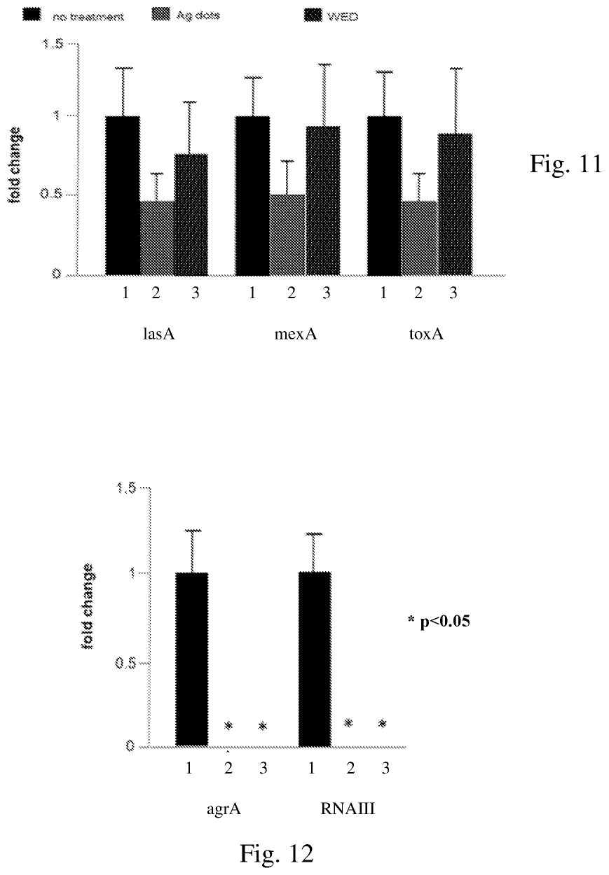

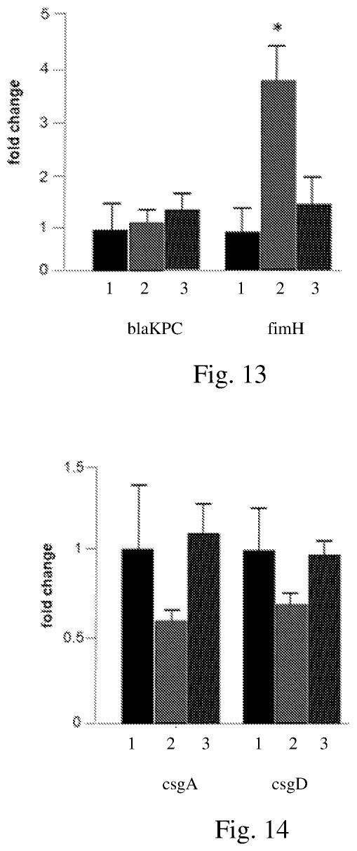

### DETAILED DESCRIPTION

### Definitions

In describing and claiming the invention, the following terminology will be used in accordance with the definitions set forth below.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

As used herein, the term “treating” includes elimination of the specific disorder or condition, or alleviation of the symptoms associated with a specific disorder or condition and/or preventing or eliminating said symptoms.

As used herein an “effective” amount or a “therapeutically effective amount” refers to a nontoxic but sufficient amount of a therapeutic treatment or pharmaceutical agent to provide the desired effect. For example one desired effect would be the or treatment of an infection by a pathogenic organism. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified RNA” is used herein to describe an RNA sequence which has been separated from other compounds including, but not limited to polypeptides, lipids and carbohydrates.

The term “isolated” requires that the referenced material be removed from its original environment (e.g., the natural environment if it is naturally occurring). For example, a naturally-occurring nucleic acid present in a living animal is not isolated, but the same nucleic acid, separated from some or all of the coexisting materials in the natural system, is isolated.

As used herein the term “patient” without further designation is intended to encompass any warm blooded vertebrate domesticated animal (including for example, but not limited to livestock, horses, mice, cats, dogs and other pets) and humans receiving therapeutic care whether or not under the direct supervision of a physician.

As used herein the term “solid support” relates to a solvent insoluble substrate that is capable of forming linkages (preferably covalent bonds) with soluble molecules. The support can be either biological in nature, such as, without limitation, a cell or bacteriophage particle, or synthetic, such as, without limitation, an acrylamide derivative, glass, plastic, agarose, cellulose, nylon, silica, or magnetized particles. The support can be in particulate form or a monolithic strip or sheet. The surface of such supports may be solid or porous and of any convenient shape.

As used herein the term “nuclease” is defined as any enzyme that can cleave the phosphodiester bonds between nucleotides of nucleic acids. The term encompasses both DNases and RNases that effect single or double stranded breaks in their target molecules. A DNase is a nuclease that catalyzes the hydrolytic cleavage of phosphodiester linkages in a DNA backbone, whereas an RNase is a nuclease that catalyzes the hydrolytic cleavage of phosphodiester linkages in an RNA backbone. The nuclease may be indiscriminate about the DNA sequence at which it cuts or alternatively, the nuclease may be sequence-specific. The nuclease may cleave only double-stranded nucleic acid, only single-stranded nucleic acid, or both double-stranded and single stranded nucleic acid. The nuclease can be an exonuclease, that cleaves nucleotides one at a time from the end of a polynucleotide chain or an endonuclease that cleaves a phosphodiester bond within a polynucleotide chain. Deoxyribonuclease I (DNase I) is an example of a DNA endonuclease that cleaves DNA (causing a double stand break) relatively nonspecifically in DNA sequences.

As used herein an antimicrobial is any agent that kills microorganisms or stops their growth, including microorganisms selected from the group consisting of bacteria, protists, and fungi.

The term “biofilm” as used herein means a community of one or more microorganisms attached to a surface, with the organisms in the community being contained within an extracellular polymeric substance (EPS) matrix produced by the microorganisms. In one embodiment the microorganism is bacterial organism. In one embodiment the biofilm is polymicrobial, containing two or more different microorganisms.

The expression “biofilm forming microorganism” encompasses any microorganism that is capable of forming a biofilm, including monomicrobial and polymicrobial biofilms.

The terms “attached” and “adhered” when used in reference to bacteria or a biofilm in reference to a surface mean that the bacteria and biofilm are established on, and at least partially coats or covers the surface, and has some resistance to removal from the surface. As the nature of this relationship is complex and poorly understood, no particular mechanism of attachment or adherence is intended by such usage.

The terms “detaching” or “removing” when used in reference to bacteria or a biofilm that is attached to a surface encompasses any process wherein a significant amount (for example at least 40%, 50%, 60%, 70%, 80% or 90%) of the bacteria or biofilm initially present on the surface is no longer attached to the surface.

As used herein the phrase “disrupting a biofilm” defines a process wherein the biofilm has been physically modified in a manner that increases the ease of dispersing and/or eliminating the microorganisms comprising the biofilm by standard procedures.

As used herein the term “adversely affecting” a biofilm, or a biofilm being “adversely affected” is intended to mean that the viability of the biofilm is compromised in some way. For example, a biofilm will be adversely affected if the number of live microorganisms that form part of the biofilm is reduced. A biofilm may also be adversely affected if its growth is inhibited, suppressed, or prevented.

As used herein the abbreviations “WED” (for wireless electroceutical device) and “BED” (for bioelectric dressing) are used interchangeably and in the absence of further characterization define any wireless electroceutical dressing or device that delivers electrical stimulation to biological tissues upon contact of those tissues in the presence of an aqueous solution.

### Embodiments

In accordance with one embodiment of the present disclosure methods are provided to mitigate the impact and prevalence of AMR microorganisms. Electrical principles influence fundamental processes of microbial organisms including adhesion to surfaces, cohesive interactions to build communities, intra and inter-species communication, and physical interactions between cells. Efflux pump activity supported by ion transport mechanisms such as voltage gated ion channels (VGICs; e.g, Kᵥ, Naᵥ etc.), enable bacteria to survive in the presence of sub-inhibitory concentrations of antibiotics until mutations for resistance emerge guaranteeing longer-term survival. Voltage-gated ion channels are also responsible for propagating long-range electrical signals within bacterial colonies. In accordance with the present disclosure chemoelectrical intervention (CEI) produces a weak electric field which is utilized in the present claimed methods to interfere with fundamental processes such a ion channel driven efflux pumps and thereby inhibit antimicrobial resistant strains and make them more susceptible to antimicrobial agents.

In accordance with one embodiment, a method of enhancing the efficacy of antimicrobial agents against an AMR strain is provided. In accordance with one embodiment the method of treating an AMR infection and/or enhancing the efficacy of antimicrobial agents against an AMR strain comprises applying an electric current or field to the AMR strain, and optionally contacting the AMR strain with an antimicrobial agent. In one embodiment the AMR strain is contacted with said antimicrobial agent simultaneously with application of the electric current or field. In one embodiment the electric current or field is applied as a series of pulses during the administration of the antimicrobial agent. In another embodiment the AMR strain is contacted with said antimicrobial agent soon after application of the electric current or field has been completed (e.g., within 1, 2, 6, 8, 12 hours after completion of the step of applying the electric current or field.

The methods disclosed herein can be applied against any pathogenic organism including a bacterial, viral, fungal, or parasitic infection. In one embodiment the AMR strain is a fungus and the antimicrobial agent is a fungicide. In one embodiment the AMR strain is an antibiotic resistant bacteria and the antimicrobial agent is an antibiotic. The use of chemoelectrical intervention (CEI) has been found to be effective against multi drug resistant microbes (MDRM) including all the bacterial strains tested (Methicillin and vancomycin resistant Staphylococcus aureus, Carbapenemase resistant Enterobacteriaceae, including Klebsiella pneumoniae, Pseudomonas aeruginosa, Acinetobacter baumannii, using CEI treatment alone or in combination with antibiotics (choice of antibiotics was determined by resistance of the strain). Such treatments significantly decreased the viability of the bacteria tested. In Candida (yeast) studies, an increased Nile red accumulation within the cell body occurred within 30 min to 3 h of treatment with CEI with or without ketoconazole compared to no treatment or ketoconazole alone.

In accordance with one embodiment the applied electric field is in the range of about 10 V/cm to about 100 V/cm, about 10 V/cm to about 50 V/cm, or about 25 V/cm to about 50 V/cm. In one embodiment the electric field is applied using a wireless electroceutical device (WED).

One embodiment of the present disclosure is directed to a method of sterilizing or reducing resident microbial organisms in an article or material that is susceptible to contamination by an AMR microorganism. In one embodiment the method comprises a step of applying a predetermined optimized electric current or field to the susceptible article.

In accordance with one embodiment a method of managing the risk of infections from an article or material that is susceptible to contamination by antimicrobial-resistant microorganisms is provided, wherein the method comprises applying a predetermined optimized electric current or field to the article or material, optionally in conjunction with administration of an antimicrobial agent. In one embodiment the AMR microorganism is a planktonic AMR organism. In one embodiment the AMR microorganism is not a biofilm. In another embodiment he AMR microorganism is one that forms a biofilm.

In accordance with one embodiment a method is provided for treating an individual in need of therapy by applying to the individual an electric field or current for a sufficient duration and under sufficient conditions, including a general site of therapy for an electric field, to provide a microbicidal or static effect to the individual and/or to the article. A sufficiently powerful source of the electric field could be provided at a distance from the site of infection. The gradient for the electric field is a weak electric field in the range of about 10 V/cm to about 100 V/cm. The article may be a material, any device implanted in the body, any device external to the body, and/or a body part. The article may be a material contacting, either directly or indirectly, a body part. The article may be a breast implant. The article may be an orthopedic implant. The microorganism may be bacteria, fungi, yeast, virus, and/or a parasite. The energy may be provided using a WED or any other form of device. The regimen provides therapeutic treatment in one embodiment. The regimen provides prophylactic treatment in one embodiment.

One embodiment is directed to a method of treating an individual having an antimicrobial-resistant microbial infection by applying a predetermined optimized electric current or field to the infection site for a duration and under conditions to facilitate therapy to the individual. In one embodiment the infection is not a biofilm. There are two kinds of bacterial strata or attachments: (i) those in which bacteria are free-floating or planktonic, and (ii) those in which bacteria are attached or sessile. The surface attachment provides additional protection for the bacteria, improves cell-cell interactions (quorum sensing), and helps concentrate nutrients. Quorum sensing is a mechanism by which bacteria detect and respond to cell population density by gene regulation. At sufficient densities, bacteria use quorum sensing to initiate nanowires, structures that electrically connect microbes and facilitate biofilm formation. CEI can be used in accordance with one embodiment to disrupt such intercellular communications.

A biofilm is a form of sessile bacteria, consisting of a dense colony of bacteria attached to a surface and defined as “a structured community of bacterial cells enclosed in a self-produced polymeric matrix and adherent to an inert or living surface,” (Costerton et al. (1999). Bacterial Biofilms: A Common Cause of Persistent Infections. Science, 284(5418), 1318-1322. doi: 10.1126/science.284.5418.1318). The polymeric matrix is connected with strong chemical bonds, and is resistant and highly adaptable to biocides, antibiotics, and physical stress. Examples of physical stress and other environmental conditions include extreme temperatures, pH changes, and exposure to ultraviolet light. Common biofilm-forming bacteria include P. aeruginosa and Staphylococcus epidermidis, both common in water, air, soil, and skin.

In one embodiment and as only one example in a larger field of application, the infection site to be treated is a wound. In one embodiment, the method is applied before any symptoms of infection, i.e., the method is applied prophylactically to prevent infection occurrence or to minimize infection severity and/or dissemination. The method may also be used in conjunction with one or more pharmaceutical agents, either over-the-counter or prescription, to treat the infection.

In the example of a wound, it will be appreciated that any type of wound may be treated by the inventive method including, but not limited to, chronic wounds, acute wounds, open wounds, closed wounds, incisions, lacerations, abrasions, etc. Illustrative examples only are pressure ulcers, diabetic foot ulcers, surgical wounds, traumatic wounds, etc.

The methods of the present disclosure encompass the use of a wireless electroceutical device (WED) to provide a source of a relatively weak electric current or field to treat an AMR microbial infection. However any device can be used to expose an AMR microbe to a therapeutic effective amount of an electric field or current that is sufficiently weak to not adversely affect human cells but effective in inhibiting or weakening AMR microbes infecting a wound or hardware. For example, the device can be electromagnetic coil with a cavity having an electric field into which, for example, a medical tool or device or a limb with an infected bone may be placed. Alternatively the patient can be exposed to a therapeutic electric field via a wearable material or fabric, a piece of clothing or cloth, a mat, an apparatus, an article such as a support, a chair, a brace, a boot, etc.

In one embodiment the electroceutical device comprises a fabric of synthetic fibers that comprises a pattern of alternating metals that form an appropriate oxidation-reduction (redox) couple for generating an electric field or current due to the transfer of an electron from one metal to the other when contacted with an aqueous solution, and in the absence of a power source. In one embodiment the electroceutical device comprises alternating dots of silver and zinc as the redox couple. In one embodiment the electroceutical device comprises a fabric of synthetic fibers having Ag dots (1-2 mm) and Zn dots (1-2 mm) printed on the fabric in proximity of about 0.5, 1, 1.5 or 2 mm to each other. In one embodiment the electroceutical device comprises a fabric of synthetic fibers having Ag dots (of about 2 mm) and Zn dots (of about 1 mm) printed on the fabric in proximity of about 0.5, 1, or 1.5 to each other. In one embodiment the electroceutical device comprises an FDA approved silver-zinc coupled bioelectric dressing (BED) which is currently being used in clinical wound care. Such a device is commercially available from Vomaris Innovations, Inc. (Tempe, AZ). The advantage of this device is that it is wireless and has no need for an external power source, can be cut to any desired shape and size, conforms to irregular surfaces, and provides an electrical field in the range of the physiologic fields (Banerjee et al, PLoS One 9(3); 2014).

The applied energy (electric field or current) disrupts the normal pathogenesis of the microbe to provide at least a microbial static effect or in many cases a microbial cidal effect. Briefly, it does so by perturbing the normal gradients within the microbial cell. Bacteria are normally very efficient in moving large amounts of ions against gradients, as subsequently described. Under normal conditions, the cell wall maintains ion gradients; without the cell wall the ions in the microbe would simply equilibrate so maintaining this differential is central to bacterial pathogenicity. The inventive method disrupts the differential and causes an insult to cell wall integrity sufficient for the microbes to enter a survival mode subsuming other functions such as infectivity.

It should be appreciated that, in some embodiments, a relatively weak electric current or field may be provided in another manner, as would be known by a person having ordinary skill in the art. For example, other techniques may be used to provide a relatively weak electric current or field such as those described in U.S. Pat. No. 8,805,522 (“Dressing for tissue treatment”), U.S. Pat. No. 9,875,340 (“Personalized pain management treatments”), U.S. Pat. No. 9,999,766 (“Device for verifying the electrical output of a microcurrent therapy device”), U.S. Pat. No. 10,309,924 (“Floating gate based sensor apparatus and related floating gate based sensor applications”), and U.S. Patent Application Publication No. 2016/0067497 (“Closed-loop vagus nerve stimulation”), the disclosure of each of which are incorporated herein by reference.

In one embodiment, the electric current or field is applied continuously. In one embodiment, the electric current or field is applied in a pulsed manner In certain embodiments, the AMR microorganism treated by the inventive method is not in the form of a biofilm.

In one embodiment, the energy applied is a relatively weak electric current or field, that is, from about 10 V/cm to about 100 V/cm. Assays to optimize electric field or current strength are known to those skilled in the art.

Various susceptible articles may be treated in accordance with methods disclosed herein. Non-limiting examples include an implantable medical device, an instrument, or a fabric that can carry the noted electric current or field. The implantable medical device may be, in non-limiting examples, an intracorporeal intraluminal device, a guidewire, a lead such as a defibrillation lead, a stent, a catheter, etc. The instrument may be, in non-limiting examples, a hemostat, forceps, a clamp, a scalpel, scissors, a pick, a retractor, a hook, a clip, pliers, a punch, a curette, a speculum, etc. The fabric may be, in non-limiting examples, a patient gown, a face mask, a glove, a scrub suit, a scrub hat, a mask, a uniform, a surgical drape, a coat, a blanket, a bandage, a dressing, a sheet, hospital linen, a pillowcase, etc. Each of these items can be prophylactically treated using the methods disclosed herein prior to use to reduce or eliminate the presence of any pathogenic microbes.

In one embodiment, the susceptible element is not limited to the material but instead could be the individual per se, or a body part of the individual. As one example, a limb could be treated by a sleeve, sock, cover, etc. As one example, a portion of the face could be treated by a face mask. As one example, the palm of a hand could be treated by a glove.

In one embodiment, the susceptible article to be treated is an implantable device. It is known that implantable devices, termed implants, may be inserted in the breast for reconstruction after mastectomy or for cosmetic breast enlargement. One example of an implant is a breast implant. Devices may be implanted in relatively healthy individuals to augment the appearance of the breast. Implants vary, as only some examples they may be textured, i.e. grooved, or non-textured, i.e., smooth, and they may be saline-filled or silicone gel-filled. Any such implanted device may be susceptible to microbial contamination, which could result in an infection for the recipient.

In breast tissue from normal healthy women, the nipple opening is populated with bacteria, and the duct between the nipple and axilla normally harbor such bacterial populations. In women with breast implants, these normal bacterial populations can escape from the confined duct and penetrate an implant surface, such as grooves, that become populated by bacteria. The normal immune response recruits immune cells, e.g., lymphocytes, to the area. Products exchanged between bacteria and lymphocytes can transform these lymphocytes into cancer cells. Left untreated, anaplastic large cell lymphoma (ALCL) may develop in the scar tissue capsule and fluid surrounding a breast implant; in some cases, it may spread throughout the body. The methods disclosed herein can address this concern. For example, the method may use a cup-like structure, bra, or other overlying material and/or device to prevent or minimize such bacterial engagement of implanted devices. Beneficially, the material or device is external, so the wearer is not subject to an additional invasive procedure.

In one embodiment, an individual with an orthopedic implant can be treated using the methods disclosed herein. In one embodiment energy is delivered by an external device into which an affected limb could be inserted, or which is externally applied to an affected site. As a non-limiting example, a cast or wrap or cover could be use on an affected limb with a power source contained in or associated with the cast, wrap, or cover. An electric field could be several inches from the affected site and still be affected. In one embodiment current is applied directly to the site. The administration duration may vary, for example, until AMR microbes are treated, or to proactively to keep an AMR microbe infection from developing.

In embodiments of the invention, the AMR microorganism may be AMR bacteria including, but not limited to, Gram-positive bacteria and Gram-negative bacteria. In addition, the AMR microorganism may be an AMR fungi, AMR parasites, AMR yeasts, etc. As examples only, the antimicrobial-resistant microorganism may be methicillin-resistant S. aureus, vancomycin-resistant S. aureus, carbapenemase-resistant K. pneumoniae, ertapenem-resistant K. pneumoniae, carbapenemase-resistant P. aeruginosa, imipenem-resistant P. aeruginosa, ampicillin-resistant S. enterica, imipenem-resistant A. baumannii, carbapenemase- resistant A. baumannii, ketoconazole-resistant C. albicans, or a multidrug-resistant C. auris. The antimicrobial-resistant microorganism may be a multidrug-resistant microorganism that is resistant to azoles, echinocandins and amphotericin B.

Another embodiment of the invention is a method to treat a subject having an antimicrobial-resistant microbial infection in a wound by applying a predetermined optimized weak electric current or field to the wound or proximate to the wound. The electric field or current may be continuous or pulsed. In certain embodiments, the antimicrobial-resistant microbial infection in a wound is not a biofilm. In embodiments of the invention, the wound includes any exudate. The method may also include the step of administering a therapeutically effective amount of an antimicrobial agent to the subject. The antimicrobial may be an antibiotic or an antifungal. The method may also include the step of administering a therapeutically effective amount of one or more other therapeutic agents to the subject. Exemplary therapeutic agents include, but are not limited to, growth factors, analgesics (e.g., an NSAID, a COX-2 inhibitor, an opioid, a glucocorticoid agent, a steroid, or a mineralocorticoid agent), anti-inflammatory agents, antiseptics (e.g., alcohol, a quaternary ammonium compound), antiproliferative agents, emollients, hemostatic agents, procoagulative agents, anticoagulative agents, immune modulators, proteins, vitamins, and the like. The antimicrobial-resistant microbe may be resistant to the antimicrobial administered. The antimicrobial-resistant microbe may be an AMR bacterium, such as an antimicrobial-resistant Gram-negative bacterium or an AMR Gram-positive bacterium or an antimicrobial-resistant yeast or an antimicrobial-resistant fungus or pathogenic virus such as the SARS-CoV-2 (COVID-19) coronavirus. The antimicrobial-resistant microbial infection may contain a microorganism such as methicillin-resistant S. aureus, vancomycin-resistant S. aureus, carbapenemase-resistant K. pneumoniae, ertapenem-resistant K. pneumoniae, carbapenemase-resistant P. aeruginosa, imipenem-resistant P. aeruginosa, ampicillin-resistant S. enterica, imipenem-resistant A. baumannii, carbapenemase-resistant A. baumannii, ketoconazole-resistant C. albicans, and a multidrug-resistant C. auris. The antimicrobial-resistant microbial infection may contain a multidrug-resistant C. auris that is resistant to azoles, echinocandins and amphotericin B. The inventive method may inhibit or disrupt microbial growth in the AMR wound infection.

In one embodiment, an electric field or electric current may be administered proactively, such as to prevent the development of AMR microbes. Alternatively, an electric field or current may be administered to treat an infection that includes antimicrobial-resistant microbes. In embodiments, an electric field or current may be delivered internally, such as from an implantable device that delivers an electric field or current imposed on body tissue surrounding the implantable device. In embodiments, an electric field or current may be delivered externally, such as from a wearable piece of clothing that contacts a body part directly or indirectly, e.g., a bra or a shoe, or any other external means for delivering an electric field. Another example of an external device is a conduit that generates an electric field or current into which an infected bodily extremity (e.g., a foot or hand) could be inserted.

The duration of exposure will vary depending on the location and severity of the infection and the device administering the electric field. With a wearable device, the hosiery, glove, bra, sock, sleeve, etc. may generally be removed at will. With an implantable device, exposure may not be controlled at will. Treatment times may range from hours to days. The method may reduce the AMR microbial load by >90% over a period of a few weeks. In one embodiment, treatment can be around four weeks. For example, the starting microbial load may be 10⁵-10⁸ colony forming units (cfu)/ml, where 10⁵ is the clinical infection threshold, and the method of treatment reduces the microbial load to below the clinical threshold in the AMR wound infection.

### EXAMPLES

The experimental design for bacteria included a viability assessment by standard growth curve for planktonic bacterial cultures, a microbial lawn assay for agar embedded wound dressings, in vitro biofilm formation on polycarbonate membrane observed by viability and staining with Syto9/propidium iodide, and scanning electron microscopy (SEM).

The experimental design for yeast utilized ketoconazole resistant Candida albicans (ATCC 64124). Methods performed included a viability assessment by standard growth curve for planktonic cultures, a microbial lawn assay for agar embedded wound dressings, a metabolism assessment by staining with [2-chloro-4-(2,3-dihydro-3-methyl-(benzo-1,3-thiazol-2-yl)-methylidene)-1-phenylquinolinium iodide] (FUN-1), an in vitro hyphae formation assay, an assay for cell membrane potential changes, an ultrastructure analysis for cell wall thickness, an in vitro biofilm formation on coverslips observed by calcofluor white/sypro ruby and confocal laser scanning microscopy (CLSM).

The experimental design for multidrug-resistant yeast included a viability assessment by standard growth curve for planktonic cultures using multidrug-resistant Candida auris from CDC FDA AR isolate bank.

The antimicrobial efficacy of a wireless electroceutical dressing (WED) was tested on antibiotic resistant bacteria. Viability assessment was by standard growth curve for planktonic cultures; microbial lawn assay for agar-embedded wound dressings; and in vitro biofilm formation on polycarbonate membrane observed by viability staining with Syto9/propidium iodide and scanning electron microscopy (SEM).

The antimicrobial efficacy of a WED was evaluated on ketoconazole resistant Candida albicans and multidrug resistant Candida auris. C. albicans pathogenic structure and function was sensitive to a weak electric field, making the resistant fungi sensitive to ketoconazole. The yeast strain tested was ketoconazole-resistant Candida albicans ATCC 64124™ (American Type Culture Collection, ATCC). Viability assessment was by standard growth curve for planktonic cultures; microbial lawn assay for agar-embedded wound dressings; metabolism assessment by FUN1 staining;

in vitro hyphae formation assay; assay for cell membrane potential changes; ultrastructure analysis for cell wall thickness; and in vitro biofilm formation on coverslips observed by Calcofluor white/Sypro Ruby and confocal laser scanning microscopy (CLSM).

ATCC strain 64124 was maintained at 4° C. on yeast extract peptone dextrose (YPD) (BD™ Difco™) agar plates. Planktonic yeast cultures (seed inoculums) were initiated in 5 ml of YPD broth (BD™ Difco™) from a single colony of the yeast strain. Cells were incubated at 28° C., under shaker conditions (200 rpm) for 24 h, followed by initiation of master inoculums for respective experiments in 5 ml YPD broth (OD₆₀₀ₙₘ ˜0.15). Yeast cells were incubated at 27° C. for all assays, except for hyphal transition experiments. For all assays, ketoconazole (Sigma Aldrich) was used at a final concentration of 100 μg/ml (dissolved in 100% methanol, stored at −20° C.).

The FDA-approved wireless electroceutical dressing (WED) Procellera (Ag—Zn dressing) (Vomaris Innovations Inc.) was the source of electric fields for treating C. albicans cells. This dressing has alternating circular regions of silver and zinc dots placed in proximity of 1 mm to each other generating electric fields in the range of 0.6-0.9 V (Banerjee et al., 2014; Banerjee et al., 2015). Polyester fabric without any metal printing was used as ‘unprinted dressing’ control to negate the effect of dressing textile; while this fabric with only silver dots was used as ‘Ag dressing’ control in all experiments, to rule out any effect due to microbicidal activity of Ag²⁺ ions. The effect of wound dressings on planktonic growth was assessed by culturing C. albicans in sterile 15 ml round bottom polypropylene tubes with 5 ml YPD broth in the presence of dressings. For dressing treatments, 2′×2′ dressings were cut and added into the culture tubes by rolling them; conducting side facing the cells. Cells were then incubated for 24 h, 48 h and 72 h at 28° C. with shaking. Culture tubes for control or untreated cells and only ketoconazole treated cells did not have any dressings. Post incubation for respective time intervals, 1 ml aliquots were taken from all groups and optical density (absorbance) was measured on a spectrophotometer at 600 nm.

An agar lawn assay assessed fungicidal activity. Dressings (2′×2′) were moistened with sterile YPD broth and placed in empty Petri plates with the conducting side of the dressing facing up. One milliliter of log phase C. albicans cells (1×10⁸ cells) was thoroughly mixed in sterile molten YPD agar (9 ml) and poured evenly in plates with dressings. For control and ketoconazole treatment, plates did not have any dressings. Post-incubation for 48 h-72 h, plates were observed for any zone of growth inhibition around dressings. Plate images were captured and the area of growth inhibition in these plates was calculated with ImageJ software. Metabolic state was assessed by FUN1 staining. The metabolic state of yeast cells, in response to wound dressings alone or with ketoconazole, was measured using a two-color fluorescent stain, FUN®1 (Invitrogen). Post treatments with electroceutical dressings, an aliquot of yeast cells (1×10⁸) was taken and washed with 1 ml of 1X PBS followed by treatment with 100 μl FUN®1 (1:1000; freshly diluted in 1X PBS) for 40 min in dark condition. Cells were then washed once with 1 ml of 1X PBS followed by counter staining with 100 μl Calcofluor White (25 μg/ml) for 10 min in dark conditions. Post counter staining cells were again washed with 1 ml 1X PBS, an aliquot of cells was trapped between a cover glass and glass slide and observed at 63X. Images captured at this magnification were analyzed using ZEN blue software to calculate the ratio of red and green fluorescence intensities for two regions of interest (ROI) per image (10 ROIs per group per technical replicate). Three technical replicates were assayed for each experimental group; six biological replicates. Cell membrane potential changes were assessed by DiBAC4(3) staining. Candida albicans cells were cultured in liquid media (YPD broth), with or without dressings or ketoconazole. At respective time intervals, an aliquot of cells (1×10⁸) was taken and washed with 1 ml 1X PBS followed by treatment with either 200 μl 10 μM DiBAC₄(3) [Bis-(1,3-dibutylbarbituric acid) trimethine oxonol] (Anaspec) [for DiBAC4(3) single stained controls] or 10 μM propidium iodide (Sigma Aldrich) [for PI single stained controls] or a combination of both stains. Heat-killed cells (1×10⁸; treated at 100° C. for 30 min) and cells treated with Amphotericin B (3 μg/ml) (Sigma

Aldrich) for 6 h were used as controls for dual stain positive and DiBAC₄(3) only positive cells, respectively.

Post staining, cells were washed with 1XPBS followed by flow cytometry (with FL1-FITC for DiBAC4(3) and FL2-PE filter for PI) (10,000 events per sample) to assess dual stain positive cells in control and test samples. For flow cytometry, control and treated cells stained only with single fluorescent stains were also run separately to check individual staining patterns. Flow cytometry data were analyzed using FlowJo software and graphically represented. Scatter plots with ratio of fluorescence intensities through FL1-FITC filter and FL2-PE filter were plotted to understand cell membrane depolarization mechanism in treated cells. The aforementioned samples remaining after flow cytometry were also observed at 63X as a microscopic confirmation. Three technical replicates were assayed for each experimental group; six biological replicates.

Ultrastructure analysis was performed as follows. After 24 h of respective treatments in YPD broth, C. albicans cells were initially fixed overnight with 500 μl 3% glutaraldehyde/0.1 M phosphate buffer; the fixative was changed to same fixative with 0.15% tannic acid for one hour. After three rinses in buffer, the specimens were post-fixed with 1% osmium tetroxide/0.1 M phosphate buffer for one hour and then rinsed multiple times with distilled water, before en bloc staining with 1% uranyl acetate in distilled water for one hour. After multiple rinses in distilled water, standard dehydration followed through a range of 70-100% ethanol, then infiltration with acetone, to 50:50 acetone and embedding resin for 48 hours. Specimens were then embedded with 100% resin (Embed 812, Electron Microscopy Sciences, Hatfield, PA) and polymerized overnight. Sections were cut, 80-85 nm and placed on copper mesh grids and viewed on Transmission Electron Microscope (TEM) (ThermoFisher, Tecnai Spirit, Hillsoboro OR) and images taken with CCD camera (Advanced Microscopy Techniques Danvers MA). Analysis of cell wall thickness was done with TEM images using ImageJ software.

Semi-quantitative analysis was performed for cell wall carbohydrates chitin, beta glucan, and mannan. After 24 h treatment, with dressings alone or in combination with ketoconazole, 1 ml yeast cells were washed once with 1 ml 1X PBS followed by chitin staining with Calcofluor White (Sigma Aldrich) (25 μg/ml) for 10 min; beta glucan staining with Aniline Blue (Anaspec) (0.05%) for 15 min; mannan staining with Concanavalin A-Texas Red conjugate (Life Technologies) (25 μg/ml) for 40 min. All stainings were performed under dark condition. Post-staining, cells were washed once with 1 ml 1X PBS and observed under 63X magnification. Microscopic images were used for measuring fluorescence intensities using ZEN blue software.

Candida albicans cells were assessed for secondary cell wall stress using the following abiotic stress agents: heat stress (42° C.), osmotic stress (1M KCl and 1M NaCl), and cell wall perturbing agents (calcofluor white—50 μg/ml and SDS—0.01%). Briefly, cells were cultured in liquid media (YPD broth), with or without dressings or ketoconazole. At respective time intervals, an aliquot was taken and cells were washed with 1 ml 1X PBS followed by adjusting the cell count to 1×10⁸ cells/ml with 1X PBS. These cells were then serially diluted (10⁻¹ to 10⁻⁶) with 1X PBS, followed by spotting 10 μl each of two selected dilutions for that time point along with the neat (undiluted) culture on YPD agar with aforementioned stressors (osmotic and cell wall perturbing agents). These plates along with control group plates (cells spotted on YPD agar plates without any stress agents) were incubated at 28° C. For high temperature stress, cells were spotted on YPD agar plates and incubated at 42° C. After 48 h-72 h incubation, plates were observed for yeast growth followed by imaging the plates and calculating the spot intensities using ImageJ software. Four technical replicates were assayed for each experimental group; eight biological replicates.

Transition of C. albicans from yeast form to hyphal form was studied with hyphae inducing conditions (YPD broth with 10% fetal bovine serum and incubation at 37° C., static condition). Cells were cultured in 5 ml hyphae-inducing medium, with or without dressings or ketoconazole. An aliquot of cells (1 ml) was taken from all treatment groups at 6 h and 24 h. Hyphal induction and elongation was monitored microscopically (63X) and imaged at 6 h and 24 h. For hyphal length measurements, aliquots were taken after 2 h in hyphae-inducing medium and images were captured at 20X magnification. Hyphal lengths were measured from these images using Accuview software (50 measurements per experimental group, six biological replicates).

In vitro Candida albicans biofilm formation in response to dressings: inhibition model. Log phase Candida albicans (1×10⁴ cells) were allowed to form biofilms, in vitro, in two sets of 24-well polystyrene microtiter plates. One set had one sterilized round cover glass (1.2 cm diameter; sterilized by treatment 100% ethanol for 10 min) in each well, while in the second set direct attachment of C. albicans biofilms on polystyrene surface was assayed using microtiter plate crystal violet assay. Control or untreated cells were provided with only YPD broth and incubated at static conditions. Treatment groups were exposed to moistened wound dressings (discs of ˜1.5 cm diameter, with conducting side facing biofilms) or ketoconazole or a combination of both with same media and incubation conditions. After every 24 h, old media was discarded and fresh YPD broth was added in all wells to replenish nutrition for growing biofilms. After 72 h, biofilms formed on cover glasses were washed thrice thoroughly with 500 μl 1X PBS to remove loosely adhered planktonic cells and fixed with 100 μl 4% paraformaldehyde for 1 hour at room temperature. Fixed biofilms were washed once with 500 μl 1X PBS and stained with 200 μl Film Tracer™ SYPRO™ Ruby (1X) (biofilm matrix stain) (Invitrogen) for 30 min followed by counterstaining with Calcofluor white (cell wall chitin binding dye; 25 μg/m1) for 10 min, to visualize yeast cells in biofilms. All staining procedures were done in dark (covered with aluminum foil) at 28° C. Post-staining biofilms were washed once again with 200 μl 1XPBS and mounted between glass slides and cover glasses with 5 μl Vectashield® Antifade mounting medium (Vector Laboratories). Biofilms were observed using confocal laser scanning microscope (at 63X) to study their spatial arrangement and thickness.

The second set of biofilms on polystyrene plates (without cover glasses) were washed thrice with 1 ml double distilled water followed by air drying and staining with 1 ml crystal violet (0.1%) (Fisher Scientific) per well for 15 min. Post-staining, plates were washed thrice with 1 ml 1X PBS, air dried and crystal violet was extracted from wells using 1 ml 90% ethanol per well. Plates were incubated with ethanol for 30 min followed by absorbance measurements at 590 nm. For scanning electron microscopy (SEM), 10 μl C. albicans cell suspension (1×10⁴ cells/ml) was spotted on 0.22 μm polycarbonate membrane discs (1 cm diameter) (GVS Lifesciences) placed on YPD agar plates (with or without ketoconazole). Once dried, the culture spots were overlaid with moistened wound dressings (˜1.2 cm diameter to cover the discs). After every 24 h, these discs were carefully transferred to fresh YPD agar plates (with or without ketoconazole). At respective time points, discs with biofilms were placed in 24 well polystyrene plates and processed for SEM. Biofilms were fixed with 2.5% glutaraldehyde in 0.1 M phosphate buffer with 50 mM sucrose (300 μl/well) at 4° C. overnight, followed by washing twice in phosphate buffer with sucrose. Biofilms were then dehydrated in graded ethanol series (50% to 100%) for 15 min each. Post-ethanol dehydrations, discs were processed with a series of ethanol: hexamethyldisilazane (HMDS) (Electron Microscopy Services) gradients (3:1, 2:2, 1:3) for 20 min each, and finally biofilms were treated with HMDS alone for 20 min and left for complete drying. Discs were then mounted on aluminum stubs and sputter coated with gold for 60 s followed by microscopic observations on SEM (JEOL 7800F, JEOL USA Inc., Peabody MA) operating at 5 kV in the secondary electron mode.

Efflux pump activity was assessed by Nile Red accumulation assay. C. albicans cells were cultured in liquid media (YPD broth), with or without dressings or ketoconazole. At respective time intervals, an aliquot of cells (1×10⁸) was taken and washed with 1 ml 1X PBS followed by treatment with an assay mixture (200 μl) of 1X PBS with 2% glucose and 7 μM Nile red (Sigma Aldrich). One set of cells was not stained and used as an unstained control, while 500 μl heat-killed cells (as treated for membrane potential assessment assays) were used as a positive control with 100% impaired efflux pump activity and showing maximum Nile red accumulation. After 30 mins of dark incubation at room temperature, cells were washed with 1 ml 1XPBS followed by flow cytometry (with FL2-PE filter) (10,000 events per sample) to assess Nile red positive cells in control and test samples. Data generated by flow cytometry were analyzed using FlowJo software and graphically represented. Scatter plots with ratio of fluorescence intensity and forward scatter were plotted to show the shift in cell populations with respect to Nile red accumulation. The aforementioned samples remaining after flow cytometry were also observed at 63X magnification as a microscopic confirmation. Three technical replicates were assayed for each experimental group; six biological replicates.

### Results

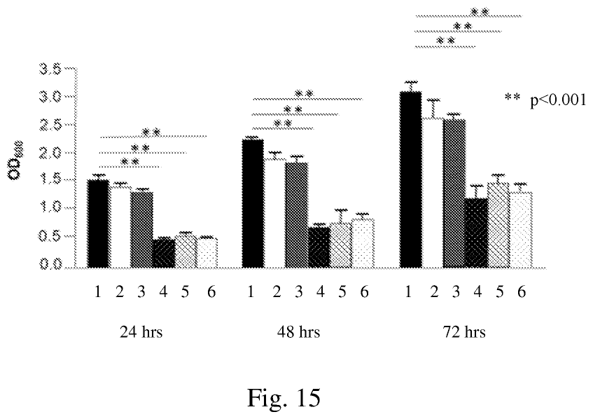

Weak electric fields impaired Candida albicans planktonic growth and metabolism. Yeast cells treated with electroceutical dressings showed a two-fold reduction in planktonic growth measured by absorbance at 600 nm (FIG. 15). This reduction was almost equivalent to the effect in ketoconazole treated cells. Planktonic growth rates in cells treated with Ag only dressing was similar to the control (untreated) and unprinted dressing treated cells. To further confirm this effect, cells were subjected to agar lawn assay with electroceutical dressings embedded in the agar layer. Even after 72 h incubation, there was a well-defined zone of growth inhibition on and around Ag—Zn dressing (˜25 cm²), while this zone was bigger when plates had Ag—Zn dressing along with a combination of ketoconazole (˜35 cm²). These results suggested synergistic activity between ketoconazole and weak electric fields.

Yeast growth was not inhibited in presence of Ag only dressing, while there was a reduction in growth when the agar lawn assay plates had ketoconazole alone. Plates without any embedded dressings or with unprinted dressing showed similar growth patterns. The metabolic state of yeast cells, in response to electroceutical dressings, alone or with ketoconazole, was measured using a two-color fluorescent stain, FUN1. Depending on plasma membrane integrity and metabolic capability, conversion of FUN1 stain by yeast cells resulted in three varied outcomes. Metabolically active cells with intact plasma membrane will convert FUN1 into orange-red or yellow-orange fluorescent intravacuolar cylindrical structures, while cells with intact plasma membrane but little or no metabolic activity will exhibit diffused green cytoplasmic fluorescence with no intravaculoar bodies. Dead cells exhibit extremely bright, diffuse, orange-red fluorescence.

As observed by FUN1 staining, control cells along with cells treated with unprinted dressing or ketoconazole showed distinct intravacuolar structures, indicating these cells had a healthy state of metabolism and intact plasma membranes. Cells treated with Ag only dressing had a mixed population of cells with intact plasma membrane integrity and reduced metabolic activity. A major population of cells treated with Ag—Zn dressings, alone or in combination with ketoconazole, showed morphological outcomes post-FUN1 staining akin to dead cells. A quantitative analysis of the ratio of red fluorescence intensity over green fluorescence intensity showed a threefold increase in this ratio in Ag-dressing alone treated cells; this ratio was even higher (˜4 fold) when cells were treated with Ag—Zn dressing along with ketoconazole. Cells treated with Ag only dressing showed a 1.5 fold decrease in this ratio compared to control cells, indicating a larger population having diffused green fluorescence in the cytoplasm. The remaining groups (control, unprinted dressing, and ketoconazole only treated cells) showed similar fluorescence intensity ratios.

Weak electric fields depolarized Candida albicans cell membrane. Cell membrane depolarization in response to weak electrical fields was assessed using the membrane potential probe DiBAC₄(3). This depolarization could be a result of either the effect on plasma membrane ion channels leading to unrestricted or uncontrolled flow of ions across the membrane, or plasma membrane damage due to pore formation resulting in passive diffusion of solutes across the compromised membrane. To determine the cause of cell membrane depolarization in response to electroceutical dressings, cells were stained with two fluorescent dyes, DiBAC₄(3) and PI. Cells stained with only one dye indicate membrane depolarization due to effect on ion channels, while cells stained with both dyes indicate membrane rupture as a reason for depolarized membrane.

Within 30 min of treatment with Ag—Zn dressing, alone or in combination with ketoconazole, ˜55% and 85% dual stain positive cells were observed by flow cytometry analysis, respectively. This trend kept increasing to ˜90% cells for both these groups after 24 h treatment. Control cells or treatment with unprinted dressing, Ag only dressing, and ketoconazole alone did not have any acute or prolonged effect on membrane depolarization. Microscopy confirmed the findings of flow cytometry analysis, indicating that Ag—Zn dressing, alone or in combination with ketoconazole, caused cell membrane damage resulting in its depolarization.

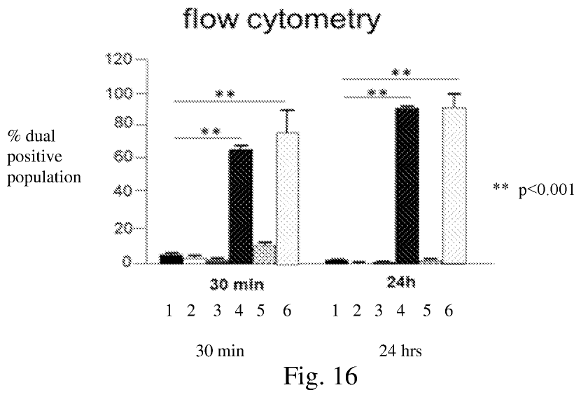

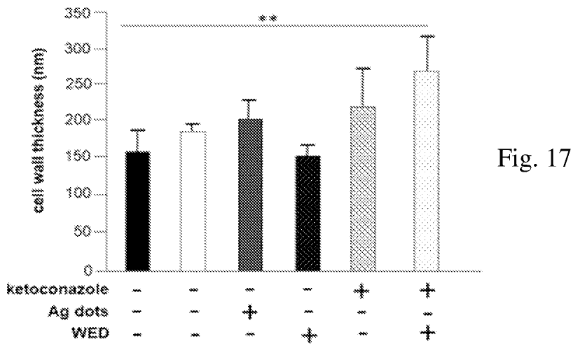

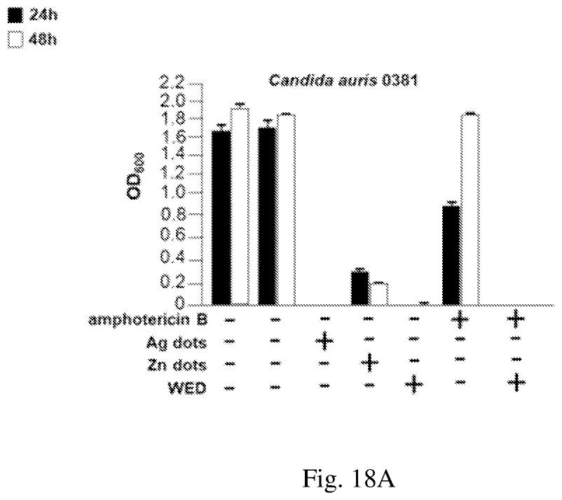

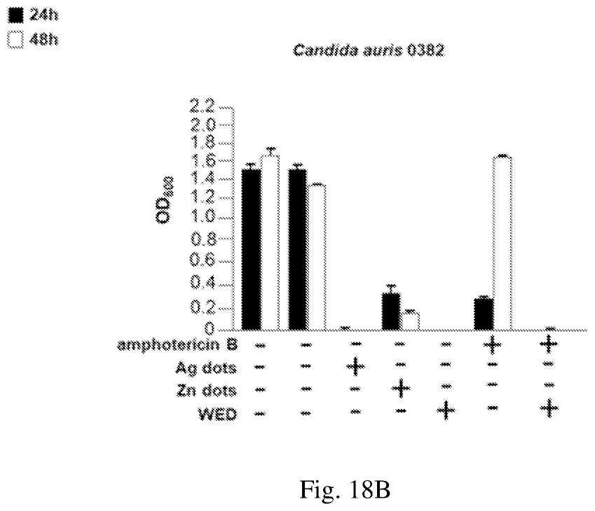

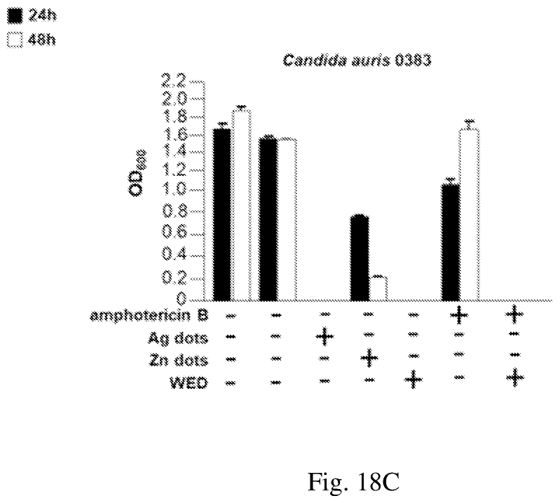

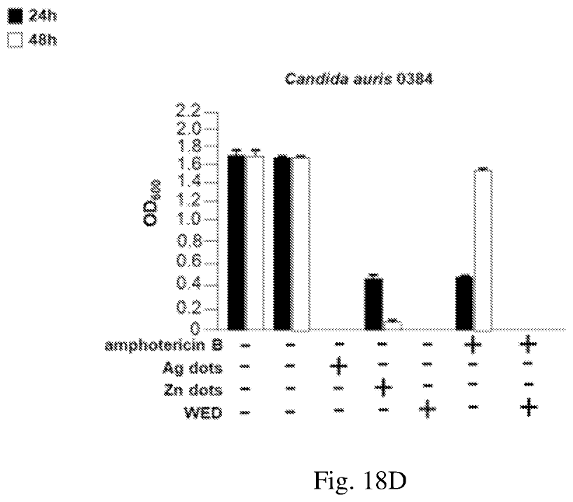

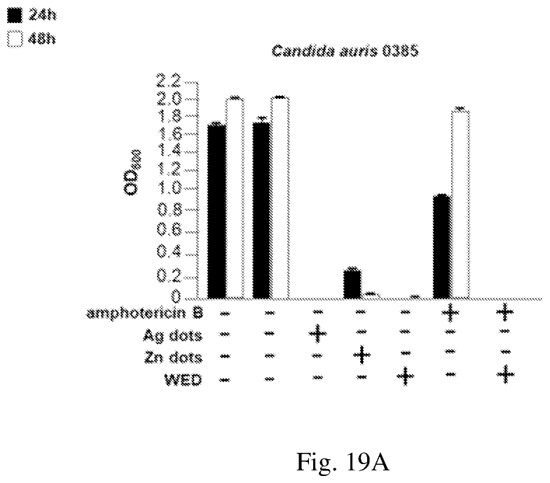

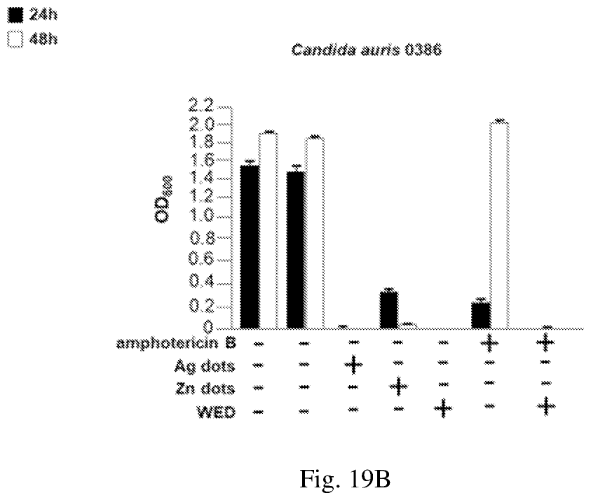

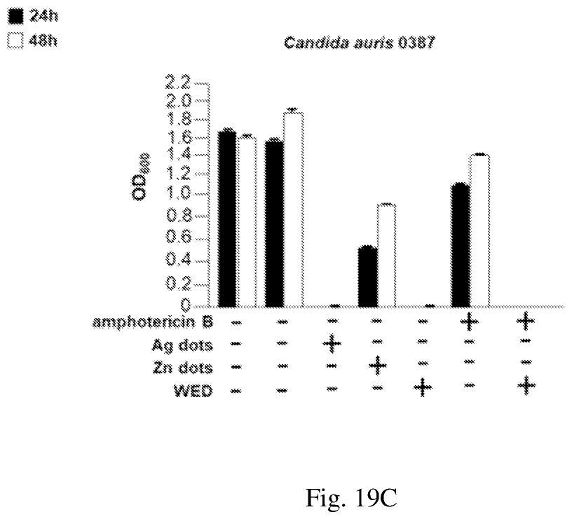

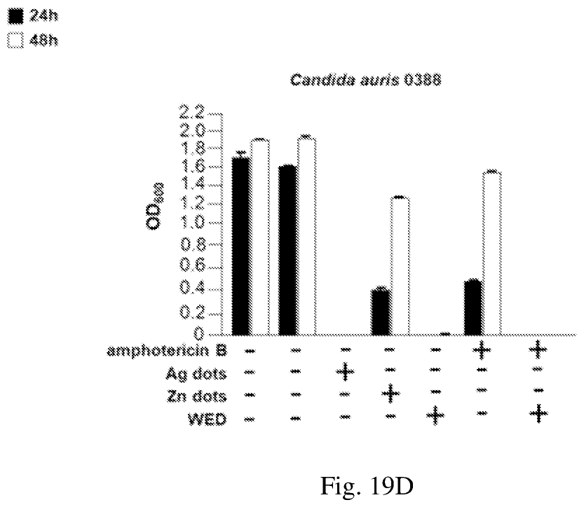

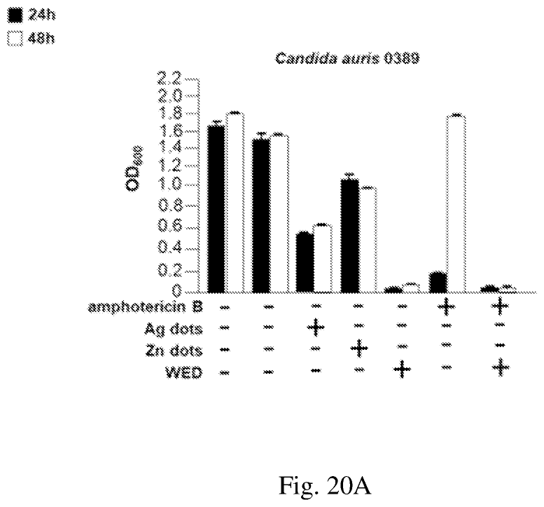

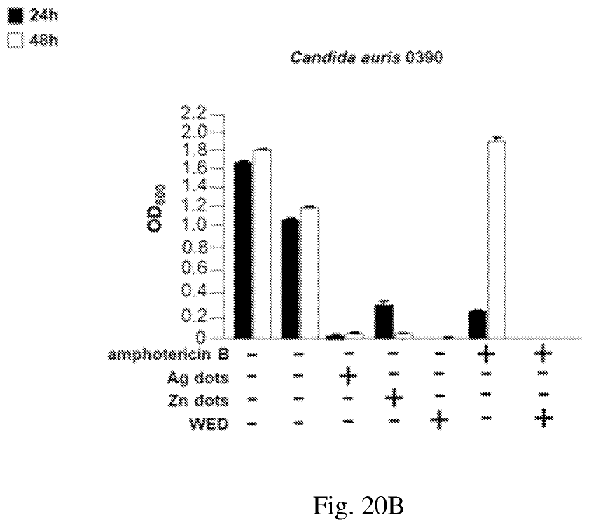

Candida albicans cell wall remodeling occurred in response to the electroceutical dressing. Cell walls act as a significant defense barrier for yeast cells, protecting from external stressors and antifungal drugs. All currently available antifungal drugs for managing infections with C. albicans are targeted towards various components of the cell wall. Since weak electric fields were applied externally to the cells, effects on cell wall remodeling or alterations was prolonged, as observed by ultrastructural studies (FIG. 16). After 24 h growth in medium with Ag—Zn dressing with ketoconazole, cells showed a two-fold increase in cell wall thickness (˜280 nm) compared to control cells or cells treated with unprinted dressing. Cells treated with ketoconazole have been reported to have thick cell walls; this effect was also observed. The Ag dressing alone in the growth medium prompted a 0.5 fold increment in cell wall thickness, while cells exposed to Ag—Zn dressing alone showed no change in thickness.

To decipher the cause for changes in cell wall thickness, electroceutical-dressing treated cells were stained with fluorescent stains specific for three main carbohydrate building blocks of the yeast cell wall: chitin (stained with Calcofluor white), glucans (stained with aniline blue), and mannans (stained with ConcanavalinA). As observed through microscopic analysis, cells exposed to Ag—Zn dressing, alone or in combination with ketoconazole, showed an increase in chitin as well as mannan content, with a significant reduction in glucan content. Cells treated with unprinted or Ag only dressing showed similar staining profiles as that of the control/untreated cells. Ketoconazole treatment alone resulted in an increased chitin staining profile. A quantitative assessment of respective fluorescence intensities corroborated with the microscopic findings for all three cell wall carbohydrates. To assess whether cell wall remodeling in response to electroceutical dressing treatment could protect the cell from any secondary stress agent, cells were first exposed to weak electrical fields followed by culturing them in presence of a secondary stressor. Exposure to secondary heat stress or osmotic stress inhibited yeast growth after 24 h, 48 h and 72 h pre-treatment with Ag—Zn dressing alone or in combination with ketoconazole. A similar effect was observed for growth in medium with Calcofluor white when cells were pre-exposed to these two experimental groups for 48 h and 72 h. The presence of SDS as a secondary stressor had a dramatic effect on yeast growth when cells were pre- treated for 1 h and 3 h with Ag—Zn dressing alone or in combination with ketoconazole, and this trend occurred until 72 h of treatment.

The morphological switch of the unicellular eukaryote Candida albicans, which exists in dimorphic states, was inhibited by weak electric fields. Yeast cell transition to a hyphal form was studied with hyphae-inducing conditions (YPD broth with 10% serum and incubation at 37° C.). Yeast cells cultured in the presence of Ag—Zn dressing alone or in combination with ketoconazole did not undergo morphological transition even after 24 h incubation in hyphae-inducing medium. This strengthens the effect of weak electrical fields in controlling the host invasive phase of the yeast life cycle. Similar observations occurred for cells treated with ketoconazole alone. Control/untreated cells or cells treated with unprinted dressing or Ag only dressing had normal hyphal induction and elongation pathways, supporting yeast to hyphal switch in these cells. Hyphal length measurements after 2 h incubation in hyphae-inducing medium showed that pseudohyphae formed in cells exposed to Ag—Zn dressing alone, ketoconazole alone, or a combination of both. Weak electric fields inhibited Candida albicans biofilm formation.

Exposing cells to weak electric fields caused cell wall remodeling and inhibited hyphal morphological transition. Both processes play an important role in biofilm formation, so effect on pathogenic morphology was probed. As SEM showed, biofilm architecture was severely affected in cells when biofilms formed in the presence of Ag—Zn dressing alone or in combination with ketoconazole, for all time points assessed (24 h, 48 h, and 72 h). Hyphal morphology was observed in biofilms of these cells only after 72 h. All other experimental groups showed proper biofilm architecture for all assessed time points). Biofilm analysis by confocal laser scanning microscopy showed a drastic reduction in biofilm thickness when cells were exposed to Ag—Zn dressing alone or in combination with ketoconazole. Similarly, these cells showed severe defects (˜5 fold reduction) in attachment to inert surfaces as measured by microtiter plate crystal violet assay.

Weak electric fields impaired Candida albicans efflux pump activity in response to wound dressings, studied by Nile red accumulation assay. Upon entry into cells, the fluorescent lipophilic dye Nile red binds to lipids and shows increased fluorescence intensity. Healthy active cells with fully functional efflux activity will efflux out Nile red when provided with glucose as a competitor in the assay conditions. Cells with impaired or less efflux activity are unable to efflux out Nile red and, when excited with an appropriate wavelength of light, exhibit increased fluorescence, which is a direct measure of Nile red accumulation. Increased Nile red accumulation within 30 min to 3 h of treatment with Ag—Zn dressing or with a combination of Ag—Zn dressing and ketoconazole indicated impaired efflux pump activity. Cells treated with ketoconazole alone showed increased efflux pump activity, in agreement with previous reports. This demonstrated that Ag—Zn dressing mediated antifungal effect on C. albicans can circumvent one of the classical antifungal resistance pathways adopted by yeast cells for their survival against antifungal drugs like ketoconazole. Microscopic analysis confirmed the flow cytometry data to show impaired efflux pump activity in cells treated with Ag—Zn dressing alone or in combination with ketoconazole. Control cells, cells treated with unprinted dressing or Ag only dressing showed a fully functional efflux pump activity with no Nile red staining.

Various modifications and additions can be made to the embodiments disclosed herein without departing from the scope of the disclosure. For example, while the embodiments described above refer to particular features, the scope of this disclosure also includes embodiments having different combinations of features and embodiments that do not include all of the described features. Thus, the scope of the present disclosure is intended to embrace all such alternatives, modifications, and variations as fall within the scope of the claims, together with all equivalents. Each of the following references is expressly incorporated by reference herein in its entirety:
Banerjee et al. (2015) Silver-Zinc Redox-Coupled Electroceutical Wound Dressing Disrupts Bacterial Biofilm. PLoS ONE 10(3): e0119531.
https://doi.org/10.1371/journal.pone.0119531
Banerjee et al. PloS One, 2014, 9(3):e89239.
Ganesh et al. (2017). Electric Field Based Dressing Disrupts Mixed-Species Bacterial

Biofilm Infection and Restores Functional Wound Healing. Annals of Surgery. 269. 1. 10.1097/SLA.0000000000002504.
Roy et al., Disposable Patterned Electroceutical Dressing (PED-10) Is Safe for Treatment of Open Clinical Chronic Wounds, Advances in Wound Care 2019 8:4, 149-159.

1. A method of enhancing the efficacy of antimicrobial agents against an antimicrobial resistant (AMR) strain, said method comprising
applying an electric current or field to the AMR strain; and
contacting the AMR strain with an antimicrobial agent.

2. The method of claim 1 wherein the AMR strain is contacted with said antimicrobial agent simultaneously with application of the electric current or field.

3. The method of claim 1 wherein the electric current or field is applied as a series of pulses.

4. The method of claim 1 wherein the AMR strain is contacted with said antimicrobial agent after application of the electric current or field has been completed.

5. The method of claim 1 wherein
i) the AMR strain is a fungus and the antimicrobial agent is a fungicide, or
ii) the AMR strain is an antibiotic resistant bacteria and the antimicrobial agent is an antibiotic.

6. (canceled)

7. The method of claim 1 wherein where the applied electric field is in the range of about 10 V/cm to about 100 V/cm.

8. The method of claim 1 wherein the electric field is applied
i) using a wireless electroceutical device (WED), or
ii) using an electromagnetic coil.

9. (canceled)

10. (canceled)

11. (canceled)

12. A method of reducing the risk of contamination by a pathogenic microorganism on an article, the method comprising
applying an electric current or field to the article, optionally in conjunction with application of an antimicrobial agent.

13. The method of claim 12 where the pathogenic microorganisms are planktonic cells.

14. The method of claim 12 wherein the article is contacted with an antimicrobial agent simultaneously with application of the electric current or field.

15. The method of claim 12 wherein the applied electric field is in the range of about 10 V/cm to about 100 V/cm.

16. The method of claim 1 wherein the electric field is applied
i) using an electromagnetic coil, or
ii) using a wireless electroceutical device (WED).

17. (canceled)

18. (canceled)

19. The method of claim 12 where the article is selected from the group consisting of a material to be placed in contact with a subject, an implantable device, a body part, and combinations thereof.

20. (canceled)

21. The method of claim 19 where the article is a device selected from the group consisting of a breast implant, an orthopedic implant, and other medical implants wherein said device is subject to an electric field prior to implantation and/or subsequent to implantation.

22. A method of treating a subject having an antimicrobial-resistant microbial infection, the method comprising
applying an electric current or field to the infected tissues to facilitate recovery from said infection in the subject.

23. The method of claim 22 further comprising administering a pharmaceutical agent having antimicrobial activity to said subject.

24. (canceled)

25. The method of claim 22 wherein
i) the AMR strain is contacted with said antimicrobial agent simultaneously with application of the electric current or field, or
ii) the AMR strain is contacted with said antimicrobial agent after application of the electric current or field has been completed.

26. (canceled)

27. The method of claim 22 wherein the AMR strain is
i) a fungus and the antimicrobial agent is a fungicide, or
ii) an antibiotic resistant bacteria and the antimicrobial agent is an antibiotic.

28. (canceled)

29. The method of claim 22 wherein the applied electric field is in the range of about 10 V/cm to about 100 V/cm, optionally wherein the electric field is applied using a wireless electroceutical device (WED).

30. (canceled)

31. The method of claim 22 where the subject has a chronic wound.
